(19) United States
(12) Patent Application Publication
(10) Pub. No.: US 2006/0009448 A1
(43) Pub. Date: Jan. 12, 2006

(54) Metabolites of 4-(3,4-dichloro-phenyl)-2-[2-(4-methyl-piperazin-1-yl)-benzylidene]thiomorpholin-3-one

(75) Inventors: Amin Mohamed Kamel, Mystic, CT (US); Kevin Albert Colizza, Mystic, CT (US); Thomas Noel O'Connell, New London, CT (US); Weiwei Wang, New Haven, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 11/135,204

(22) Filed: May 23, 2005

Related U.S. Application Data
(60) Provisional application No. 60/573,548, filed on May 21, 2004.

(51) Int. Cl. A61K 31/541; C07D 417/02

(52) U.S. Cl. 514/227.5; 544/60

(57) ABSTRACT

Metabolites of 4-(3,4-dichloro-phenyl)-2-[2-(4-methyl-piperazin-1-yl)-benzylidene]-thiomorpholin-3-one, and use of same.

### FIELD OF THE INVENTION

The invention relates to compounds that are mammalian metabolites of 4-(3,4-dichloro-phenyl)-2-[2-(4-methyl-piperazin-1-yl)-benzylidene]-thiomorpholin-3-one, and pharma-ceutical compositions thereof, as well as use of same as therapeutic agents in e.g. methods for treatment of diseases associated with migraine, depression for which a 5-HT₁ antagonist is indicated, and as analytical assay standards.

### BACKGROUND OF THE INVENTION

Antagonists of serotonin 1 (5-HT₁) receptors, specifically, of one or both of the 5-HT_{1A} and 5-HT_{1D} receptors are useful in treating hypertension, all forms of depression (e.g., depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition), bipolar disorder (including in the depressed phase), generalized anxiety disorder, social anxiety, separation anxiety disorder, phobias (e.g., agoraphobia, social phobia and simple phobias), posttraumatic stress syndrome, avoidant personality disorder, premature ejaculation, eating disorders (e.g., binge eating disorder, anorexia nervosa and bulimia nervosa), obesity, chemical dependencies (e.g., addictions to alcohol, cocaine, heroin, phenobarbital, marijuana, nicotine and benzodiazepines), cluster headache, migraine, pain, Alzheimer's disease, obsessive-compulsive disorder; panic disorder with and without agoraphobia; memory disorders (e.g., dementia, amnestic disorders, and age-related cognitive decline (ARCD)), Parkinson's diseases (e.g., dementia in Parkinson's disease, neuroleptic-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania, headache (associated with vascular disorders) autism, pervasive developmental disorder NOS, Asperger's disorder, selective mutism, chronic motor or vocal tic disorder, somatization disorder, insomnia, intermittent explosive disorder, pyromania, pathological gambling, impulse-control disorder, premenstrual dysphoric disorder, and attention-deficit/hyperactivity disorder (ADHD), and other disorders for which a 5-HT, agonist or antagonist is indicated.

European Patent Publication 434,561, published on Jun. 26, 1991, refers to 7-alkyl, alkoxy, and hydroxy substituted-1-(4-substituted-1-piperazinyl)-naphthalenes. The compounds are referred to as 5-HT₁ agonists and antagonists useful for the treatment of migraine, depression, anxiety, schizophrenia, stress and pain.

European Patent Publication 343,050, published on Nov. 23, 1989, refers to 7-unsubstituted, halogenated, and methoxy substituted-1-(4-substituted-1-piper-azinyl) naphthalenes as useful 5-HT_{1A} ligand therapeutics.

PCT publication WO 94/21619, published Sep. 29, 1994, refers to naphthalene derivatives as 5-HT₁ agonists and antagonists.

PCT publication WO 96/00720, published Jan. 11, 1996, refers to naphthyl ethers as useful 5-HT₁ agonists and antagonists.

European Patent Publication 701,819, published Mar. 20, 1996, refers to the use of 5-HT₁ agonists and antagonists in combination with a 5-HT re-uptake inhibitor.

Glennon et al., refers to 7-methoxy-1-(1-piperazinyl)-naphthalene as a useful 5-HT₁ ligand in their article “5-HT_{1D} Serotonin Receptors”, Drug Dev. Res., 22, 25-36 (1991).

Glennon's article “Serotonin Receptors: Clinical Implications”, Neuroscience and Behavioral Reviews, 14, 35-47 (1990), refers to the pharmacological effects associated with serotonin receptors including appetite suppression, thermoregulation, cardiovascular/hypotensive effects, sleep, psychosis, anxiety, depression, nausea, emesis, Alzheimer's disease, Parkinson's disease and Huntington's disease.

World Patent Application WO 95/31988, published Nov. 30, 1995, refers to the use of a 5-HT_{1D} antagonist in combination with a 5-HT_{1A} antagonist to treat CNS disorders such as depression, generalized anxiety, panic disorder, agoraphobia, social phobias, obsessive-compulsive disorder, post-traumatic stress disorder, memory disorders, anorexia nervosa and bulimia nervosa, Parkinson's disease, tardive dyskinesias, endocrine disorders such as hyperprolactinaemia, vasospasm (particularly in the cerebral vasculature) and hypertension, disorders of the gastrointestinal tract where changes in motility and secretion are involved, as well as sexual dysfunction.

G. Maura et al., J. Neurochem, 66 (1), 203-209 (1996), have stated that administration of agonists selective for 5-HT_{1A} receptors or for both 5-HT_{1A} and 5-HT_{1D} receptors might represent a great improvement in the treatment of human cerebellar ataxias, a multifaceted syndrome for which no established therapy is available.

European Patent Publication 666,261, published Aug. 9, 1995 refers to thiazine and thiomorpholine derivatives which are claimed to be useful for the treatment of cataracts.

### SUMMARY OF THE INVENTION

In one aspect, the invention relates to a purified and isolated metabolite of 4-(3,4-dichloro-phenyl)-2-[2-(4-methyl-piperazin-1-yl)-benzylidene]-thiomorpholin-3-one having Formula I. In a preferred aspect, the invention relates to a metabolite having Formulae II or III. The invention also relates to a pharmaceutical composition employing one or more of said metabolites; a method of treating a disease for which a 5-HT₁ antagonist is indicated using one or more of said metabolites, and as an assay employing said metabolite as a standard.

### DETAILED DESCRIPTION OF THE INVENTION

In one practice, and without limitation, the invention relates to a purified and isolated metabolite of 4-(3,4-dichloro-phenyl)-2-[2-(4-methyl-piperazin-1-yl)-benzylidene]-thiomorpholin-3-one having Formula (I): 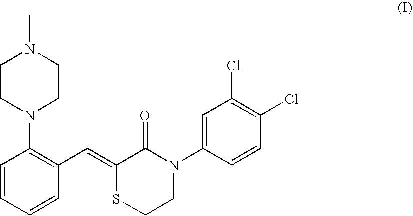
and the racemic-diastereomeric mixtures and optical isomers thereof, the prodrugs thereof, and the pharmaceutiacily acceptable salts of said metabolites, racemic-diastereomeris mixtures, optical isomers, and prodrugs.

In another embodiment, the invention relates to a purified and isolated metabolite having Formulae (II): 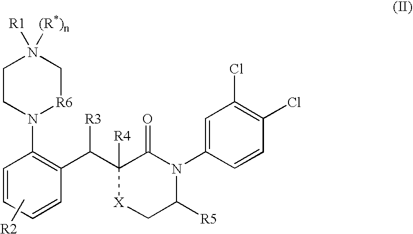
wherein R1 is H or CH₃;
R2 is H, CH₃, or —O—Gluc;
R3 is H or OH, or R3 and R4 form a bond, or R3 and R6 together with the C and N atoms to which they are respectively attached form a 5-membered unsaturated ring, or R3 and R4 together are —O— and form a 3-membered epoxide ring with the C atom to which they are respectively attached;
R4 is H or CH₃ when X is S, or R4 is (═O) when X is SH;
R5 is H, OH or (═O);
R6 is —CH₂—;
X is S when bond (—) is present, and SH when bond (—) is absent;
n is 0 or 1;
R* is O or is a bond between the N atom to which it is attached and an adjacent C atom whereby in either case said N atom has a positive charge; and the racemic-diastereomeric mixtures and optical isomers thereof, the prodrugs thereof, and the pharmaceutically acceptable salts of said metabolites, racemic-diastereomer mixtures, optical isomers, and prodrugs; with the proviso that R3 and R4 do not form a bond when R1 is CH₃, R6 is CH₂ and R2 and R3 are each hydrogen.

In another embodiment, the invention relates to a purified and isolated metabolite having Formulae (III): 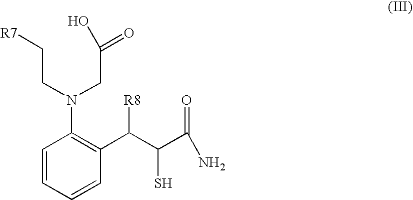
wherein:
R7 is NH₂ or OH;
R8 is H or OH; and
the racemic-diastereomeric mixtures and optical isomers thereof, the prodrugs thereof, and the pharmaceutically acceptable salts of said metabolites, racemic-diastereomer mixtures, optical isomers, and prodrugs.

Preferably, the purified and isolated metabolite of the above Formulae (II) and (III) is selected from the group consisting of Formulae (IV) to (XXIV):

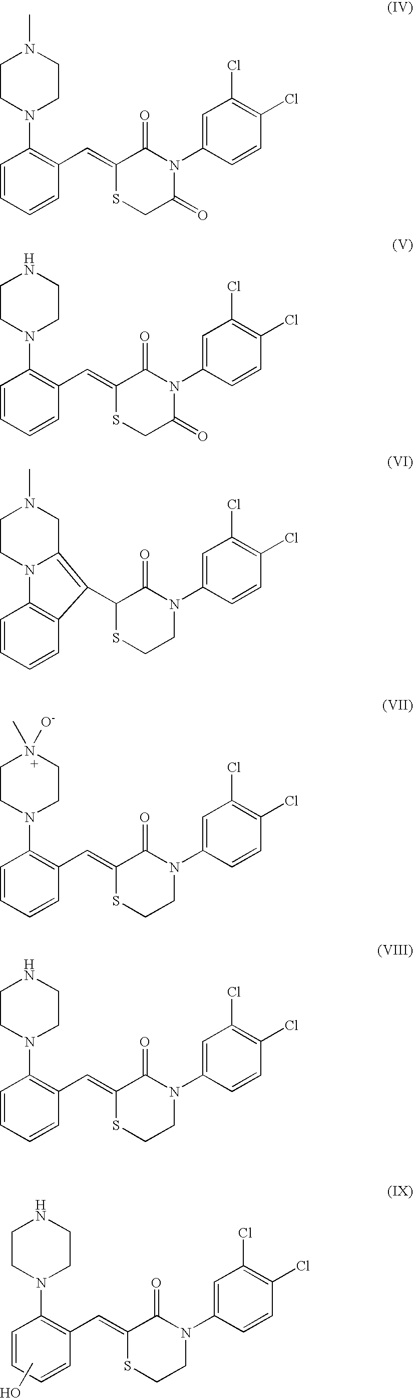

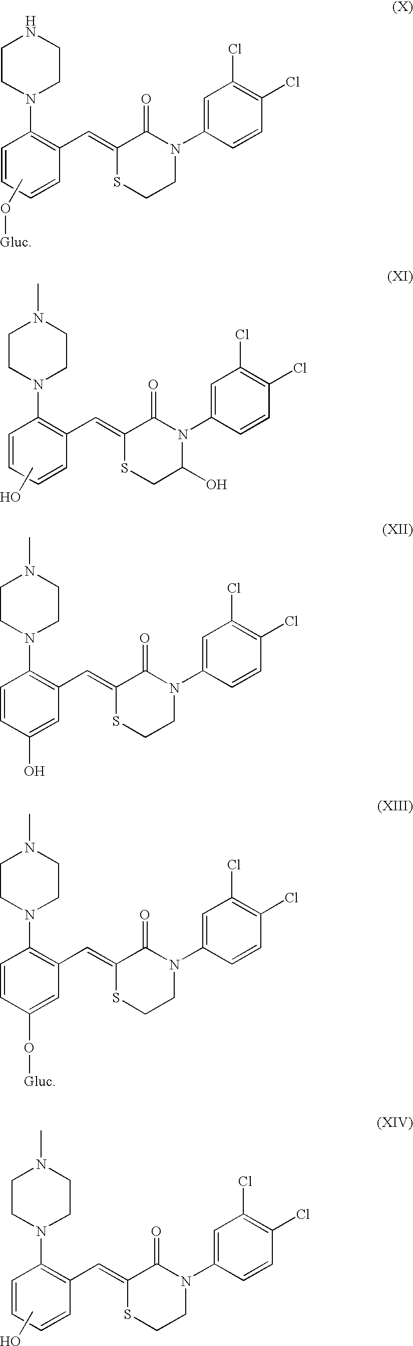

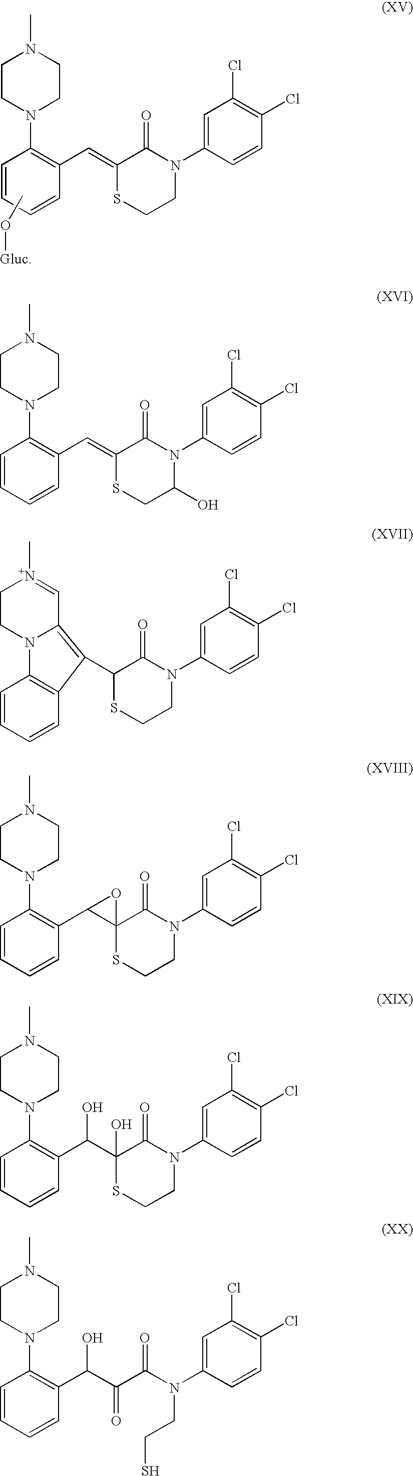

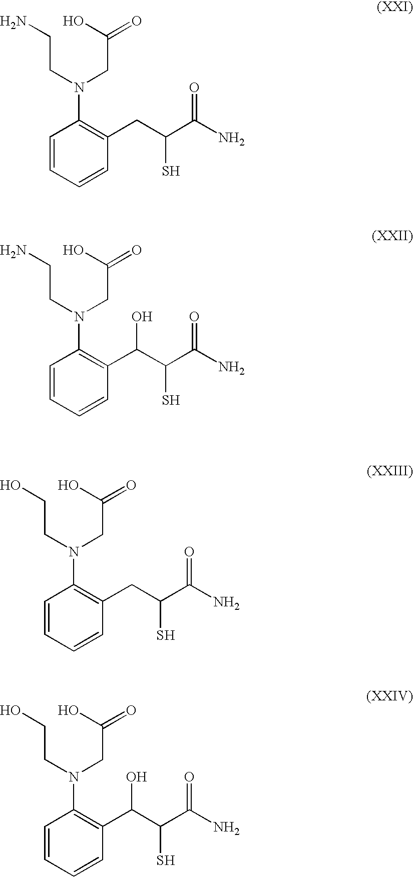

In other aspects, the invention relates to a pharmaceutical composition comprising one or more metabolites of Formula (I) and a pharmaceutically acceptable carrier; preferably one or more metabolites of Formulae (II) and/or (III) and a pharmaceutically acceptable carrier; more preferably one or more metabolites of Formulae (IV) to (XXIV) individually or in any combination thereof, and a pharmaceutically acceptable carrier.

In another practice, the invention relates to a pharmaceutical composition comprising one or more metabolites of formula I and a pharmaceutically acceptable carrier.

In another practice, the invention relates to an assay for assessing the metabolic fate of 4-(3,4-dichloro-phenyl)-2-[2-(4-methyl-piperazin-1-yl)-benzylidene]-thiomorpholin-3-one, said assay comprising the metabolite of Formula (I); preferably Formulae (II) and/or (III); more preferably Formulae (IV) to (XXIV) individually or in any combination thereof.

The chemist of ordinary skill will recognize that certain compounds of this invention will contain one or more atoms which can be in a particular stereochemical, tautomeric, or geometric configuration, giving rise to stereoisomers, tautomers, regio and configurational isomers. All such isomers and mixtures thereof are included in this invention. Hydrates and solvates of the compounds of this invention are also included.

The subject invention also includes isotopically-labeled compounds, which are identical to those shown in Formulae I-XXIV, among other compounds encompassed by the invention, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, sulfur, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively.

Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example, those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., H, can afford certain therapeutic advantages resulting from greater metabolic stability, for example increased in vivo half-life or reduced dosage requirements and, hence, can be preferred in some circumstances. Isotopically labeled compounds of formulae I-XXV of this invention and prodrugs thereof can generally be prepared by carrying out the procedures exemplified below or those known in the art. ¹⁴C-4-(3,4-dichloro-phenyl)-2-[2-(4-methyl-piperazin-1-yl)-benzylidene]-thiomorpholin-3-one can be prepared by the methods outlined and exemplified in U.S. Pat. No. 5,552,412 by substituting a readily available isotopically labeled reagent for a non-isotopically labeled reagent.

The metabolites of 4-(3,4-dichloro-phenyl)-2-[2-(4-methyl-piperazin-1-yl)-benzylidene]-thiomorpholin-3-one, in their substantially pure form or in mixtures of known composition, can be used as analytical standards for in vitro or in vivo metabolism studies or as intermediates for the chemical synthesis or biosynthesis of new chemical entities. The metabolites can be isolated as solids or in solutions.

In another practice, the invention relates to a pharmaceutical composition for treating a disorder or condition selected from hypertension, all forms of depression (e.g., depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition), bipolar disorder (including in the depressed phase), generalized anxiety disorder, social anxiety, separation anxiety disorder, phobias (e.g., agoraphobia, social phobia and simple phobias), posttraumatic stress syndrome, avoidant personality disorder, premature ejaculation, eating disorders (e.g., binge eating disorder, anorexia nervosa and bulimia nervosa), obesity, chemical dependencies (e.g., addictions to alcohol, cocaine, heroin, phenobarbital, marijuana, nicotine and benzodiazepines), cluster headache, migraine, pain, Alzheimer's disease, obsessive-compulsive disorder; panic disorder with and without agoraphobia; memory disorders (e.g., dementia, amnestic disorders, and age-related cognitive decline (ARCD)), Parkinson's diseases (e.g., dementia in Parkinson's disease, neuroleptic-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania, headache (associated with vascular disorders) autism, pervasive developmental disorder NOS, Asperger's disorder, selective mutism, chronic motor or vocal tic disorder, somatization disorder, insomnia, intermittent explosive disorder, pyromania, pathological gambling, impulse-control disorder, premenstrual dysphoric disorder, and attention-deficit/hyperactivity disorder (ADHD), in a mammal, preferably a human, comprising an amount of one or more metabolites of formula I or a pharmaceutically acceptable salt thereof effective in treating such disorder or condition and a pharmaceutically acceptable carrier.

In another practice, the invention relates to a pharmaceutical composition for treating a disorder or condition that can be treated by enhancing serotonergic neurotransmission in a mammal, preferably a human, comprising an amount of one or more metabolites of formula I, or a pharmaceutically acceptable salt thereof, effective in treating such disorder or condition and a pharmaceutically acceptable carrier. Examples of such disorders and conditions are those enumerated in the preceding paragraph.

In another practice, the invention relates to a pharmaceutical composition for treating a disorder or condition selected from attention-deficit/hyperactivity disorder (ADHD), bipolar disorder, bipolar disorder-depressed phase; mild, moderate, or severe depression with or without atypical features, melancholic features, psychotic features, catatonic features; seasonal affective disorder, postpartum depression, geriatric depression, chronic depression, dysthymia, adjustment disorder with depressed mood, adjustment disorder with anxiety, depressed mood, mixed anxiety and depression, substance induced mood disorder, mood disorder secondary to a general medical condition, social anxiety, separation anxiety disorder, binge eating disorder, dependencies and addictions to marijuana, panic disorder with agoraphobia; autism, pervasive developmental disorder NOS, Asperger's disorder, selective mutism, chronic motor or vocal tic disorder, somatization disorder, insomnia, intermittent explosive disorder, pyromania, pathological gambling, impulse-control disorder, and premenstrual dysphoric disorder, in a mammal, preferably a human, comprising an amount of one or more metabolites of formula I or a pharmaceutically acceptable salt thereof effective in treating such disorder or condition and a pharmaceutically acceptable carrier.

In another practice, the invention relates to a method for treating a disorder or condition selected from hypertension, all forms of depression (e.g., depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition), bipolar disorder (including in the depressed phase), generalized anxiety disorder, social anxiety, separation anxiety disorder, phobias (e.g., agoraphobia, social phobia and simple phobias), posttraumatic stress syndrome, avoidant personality disorder, premature ejaculation, eating disorders (e.g., binge eating disorder, anorexia nervosa and bulimia nervosa), obesity, chemical dependencies (e.g., addictions to alcohol, cocaine, heroin, phenobarbital, marijuana, nicotine and benzodiazepines), cluster headache, migraine, pain, Alzheimer's disease, obsessive-compulsive disorder; panic disorder with and without agoraphobia; memory disorders (e.g., dementia, amnestic disorders, and age-related cognitive decline (ARCD)), Parkinson's diseases (e.g., dementia in Parkinson's disease, neuroleptic-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania, headache (associated with vascular disorders) autism, pervasive developmental disorder NOS, Asperger's disorder, selective mutism, chronic motor or vocal tic disorder, somatization disorder, insomnia, intermittent explosive disorder, pyromania, pathological gambling, impulse-control disorder, premenstrual dysphoric disorder, and attention-deficit/hyperactivity disorder (ADHD), in a mammal, preferably a human, comprising administering to a mammal in need of such treatment an amount of one or more metabolites of formula I, or a pharmaceutically acceptable salt thereof, that is effective in treating such disorder or condition.

In another practice, the invention relates to a method for treating a disorder or condition selected from attention-deficit/hyperactivity disorder (ADHD), bipolar disorder, bipolar disorder-depressed phase; mild, moderate, or severe depression with or without atypical features, melancholic features, psychotic features, catatonic features; seasonal affective disorder, postpartum depression, geriatric depression, chronic depression, dysthymia, adjustment disorder with depressed mood, adjustment disorder with anxiety, depressed mood, mixed anxiety and depression, substance induced mood disorder, mood disorder secondary to a general medical condition, social anxiety, separation anxiety disorder, binge eating disorder, dependencies and addictions to marijuana, panic disorder with agoraphobia; autism, pervasive developmental disorder NOS, Asperger's disorder, selective mutism, chronic motor or vocal tic disorder, somatization disorder, insomnia, intermittent explosive disorder, pyromania, pathological gambling, impulse-control disorder, and premenstrual dysphoric disorder, in a mammal, preferably a human, comprising administering to a mammal in need of such treatment an amount of one or more metabolites of formula I, or a pharmaceutically acceptable salt thereof, that is effective in treating such disorder or condition.

In another practice, the invention relates to a method for treating a disorder or condition that can be treated by enhancing serotonergic neurotransmission in a mammal, preferably a human, comprising administering to a mammal in need of such treatment an amount of one or more metabolites of formula I, or a pharmaceutically acceptable salt thereof, that is effective in treating such disorder or condition.

In another practice, the invention relates to a pharmaceutical composition for treating a disorder or condition selected from hypertension, all forms of depression (e.g., depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition), bipolar disorder (including in the depressed phase), generalized anxiety disorder, social anxiety, separation anxiety disorder, phobias (e.g., agoraphobia, social phobia and simple phobias), posttraumatic stress syndrome, avoidant personality disorder, premature ejaculation, eating disorders (e.g., binge eating disorder, anorexia nervosa and bulimia nervosa), obesity, chemical dependencies (e.g., addictions to alcohol, cocaine, heroin, phenobarbital, marijuana, nicotine and benzodiazepines), cluster headache, migraine, pain, Alzheimer's disease, obsessive-compulsive disorder; panic disorder with and without agoraphobia; memory disorders (e.g., dementia, amnestic disorders, and age-related cognitive decline (ARCD)), Parkinson's diseases (e.g., dementia in Parkinson's disease, neuroleptic-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania, headache (associated with vascular disorders) autism, pervasive developmental disorder NOS, Asperger's disorder, selective mutism, chronic motor or vocal tic disorder, somatization disorder, insomnia, intermittent explosive disorder, pyromania, pathological gambling, impulse-control disorder, premenstrual dysphoric disorder, and attention-deficit/hyperactivity disorder (ADHD), in a mammal, preferably a human, comprising a serotonin receptor antagonizing or agonizing effective amount of one or more metabolites of formula I, or a pharmaceutically acceptable salt thereof, and a pharmaceutically acceptable carrier.

In another practice, the invention relates to a pharmaceutical composition for treating a disorder or condition that can be treated by enhancing serotonergic neurotransmission in a mammal, preferably a human, comprising a serotonin receptor antagonizing or agonizing effective amount of one or more metabolites of formula I, or a pharmaceutically acceptable salt thereof, and a pharmaceutically acceptable carrier.

In another practice, the invention relates to a pharmaceutical composition for treating a disorder or condition selected from attention-deficit/hyperactivity disorder (ADHD), bipolar disorder, bipolar disorder-depressed phase; mild, moderate, or severe depression with or without atypical features, melancholic features, psychotic features, catatonic features; seasonal affective disorder, postpartum depression, geriatric depression, chronic depression, dysthymia, adjustment disorder with depressed mood, adjustment disorder with anxiety, depressed mood, mixed anxiety and depression, substance induced mood disorder, mood disorder secondary to a general medical condition, social anxiety, separation anxiety disorder, binge eating disorder, dependencies and addictions to marijuana, panic disorder with agoraphobia; autism, pervasive developmental disorder NOS, Asperger's disorder, selective mutism, chronic motor or vocal tic disorder, somatization disorder, insomnia, intermittent explosive disorder, pyromania, pathological gambling, impulse-control disorder, and premenstrual dysphoric disorder, in a mammal, preferably a human, comprising a serotonin receptor antagonizing or agonizing effective amount of one or more metabolites of formula I, or a pharmaceutically acceptable salt thereof, and a pharmaceutically acceptable carrier.

In another practice, the present invention relates to a method for treating a disorder or condition selected from hypertension, all forms of depression (e.g., depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition), bipolar disorder (including in the depressed phase), generalized anxiety disorder, social anxiety, separation anxiety disorder, phobias (e.g., agoraphobia, social phobia and simple phobias), posttraumatic stress syndrome, avoidant personality disorder, premature ejaculation, eating disorders (e.g., binge eating disorder, anorexia nervosa and bulimia nervosa), obesity, chemical dependencies (e.g., addictions to alcohol, cocaine, heroin, phenobarbital, marijuana, nicotine and benzodiazepines), cluster headache, migraine, pain, Alzheimer's disease, obsessive-compulsive disorder; panic disorder with and without agoraphobia; memory disorders (e.g., dementia, amnestic disorders, and age-related cognitive decline (ARCD)), Parkinson's diseases (e.g., dementia in Parkinson's disease, neuroleptic-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania, headache (associated with vascular disorders) autism, pervasive developmental disorder NOS, Asperger's disorder, selective mutism, chronic motor or vocal tic disorder, somatization disorder, insomnia, intermittent explosive disorder, pyromania, pathological gambling, impulse-control disorder, premenstrual dysphoric disorder, and attention-deficit/hyperactivity disorder (ADHD), in a mammal, preferably a human, comprising administering to a mammal requiring such treatment a serotonin receptor antagonizing or agonizing effective amount of one or more metabolites of formula I or a pharmaceutically acceptable salt thereof.

In another practice, the invention relates to a method for treating a disorder or condition that can be treated by enhancing serotonergic neurotransmission in a mammal, preferably a human, comprising administering to a mammal requiring such treatment a serotonin receptor antagonizing or agonizing effective amount of one or more metabolites of formula I or a pharmaceutically acceptable salt thereof.

In another practice, the invention relates to a method for treating a disorder or condition selected from attention-deficit/hyperactivity disorder (ADHD), bipolar disorder, bipolar disorder-depressed phase; mild, moderate, or severe depression with or without atypical features, melancholic features, psychotic features, catatonic features; seasonal affective disorder, postpartum depression, geriatric depression, chronic depression, dysthymia, adjustment disorder with depressed mood, adjustment disorder with anxiety, depressed mood, mixed anxiety and depression, substance induced mood disorder, mood disorder secondary to a general medical condition, social anxiety, separation anxiety disorder, binge eating disorder, dependencies and addictions to marijuana, panic disorder with agoraphobia; autism, pervasive developmental disorder NOS, Asperger's disorder, selective mutism, chronic motor or vocal tic disorder, somatization disorder, insomnia, intermittent explosive disorder, pyromania, pathological gambling, impulse-control disorder, and premenstrual dysphoric disorder, in a mammal, preferably a human, comprising administering to a mammal requiring such treatment a serotonin receptor antagonizing or agonizing amount of one or more metabolites of formula I, or a pharmaceutically acceptable salt thereof.

The compounds of the present invention are also useful in the treatment of patients afflicted with two or more of the above disorders. It is not uncommon for certain of the above listed disorders, which can be treated using the novel compounds of the invention, to exist in patients afflicted with one or more other such disorders. For example, depression is often comorbid with anxiety and both can be treated using the compounds or pharmaceutical compositions of the present invention.

A further particular advantage of the serotonin 1 (5-HT₁) receptor agonist/antagonist compounds of the present invention is that they exhibit pharmacological and therapeutic activity without the delayed onset of action usually associated with selective serotonin reuptake inhibitors.

In another practice, the invention relates to a pharmaceutical composition for treating a condition or disorder that can be treated by enhancing serotonergic neurotransmission in a mammal, preferably a human, comprising:
a) a pharmaceutically acceptable carrier;
b) one or more metabolites of formula I or a pharmaceutically acceptable salt thereof; and
c) one or more 5-HT re-uptake inhibitors, preferably sertraline, or a pharmaceutically acceptable salt thereof;
wherein the amount of the active compounds (i.e., the compound of formula I and the 5-HT re-uptake inhibitor) are such that the combination is effective in treating such disorder or condition.

In another practice, the invention relates to a method for treating a disorder or condition that can be treated by enhancing serotonergic neurotransmission in a mammal, preferably a human, comprising administering to a mammal requiring such treatment:
a) one or more metabolites of formula I, defined above, or a pharmaceutically acceptable salt thereof; and
b) one or more 5-HT re-uptake inhibitors, preferably sertraline, or a pharmaceutically acceptable salt thereof;
wherein the amounts of the active compounds (i.e., the compound of formula I and the 5-HT re-uptake inhibitor) are such that the combination is effective in treating such disorder or condition.

In another practice, the invention relates to a method for treating a disorder or condition that can be treated by enhancing serotonergic neurotransmission in a mammal, preferably a human, comprising administering to said mammal requiring such treatment:
a) one or more 5-HT_{1A} antagonists or a pharmaceutically acceptable salt thereof; and
b) one or more 5-HT_{1D} antagonists of formula I or a pharmaceutically acceptable salt thereof;
wherein the amounts of each active compound (i.e., the 5-HT_{1A} antagonist and the 5-HT_{1D} antagonist) are such that the combination is effective in treating such disorder or condition.

In another practice, the invention relates to a pharmaceutical composition for treating a disorder or condition that can be treated by enhancing serotonergic neurotransmission in a mammal, preferably a human, comprising:
a) one or more 5-HT_{1A} antagonists or a pharmaceutically acceptable salt thereof; and
b) one or more 5-HT_{1D} antagonists of formula I or a pharmaceutically acceptable salt thereof;
wherein the amounts of each active compound (i.e., the 5-HT_{1A} antagonist and the 5-HT_{1D} antagonist) are such that the combination is effective in treating such disorder or condition.

Sertraline, (1S-cis)-4-(3,4-dichlorophenyl)-1,2,3,4-tetrahydro-N-methyl-1-naphthalenamine, as used herein has the following structural formula 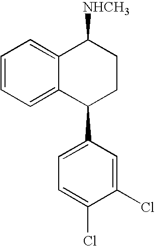
and ordinarily used in the form of its hydrochloride salt. The synthesis of sertraline is described in U.S. Pat. No. 4,536,518, assigned to Pfizer Inc. Sertraline hydrochloride is useful as an antidepressant and anorectic agent, and is also useful in the treatment of depression, chemical dependencies, anxiety obsessive compulsive disorders, phobias, panic disorder, post traumatic stress disorder, and premature ejaculation.

In the methods of treatment of the present invention, a metabolite can be administered to a subject directly, such as in a tablet, or the metabolite can be administered by being produced in the subject's body through metabolism. For example, a metabolite of the present invention can be effectively administered to a subject to treat a disease or condition by administering to the subject an amount of 4-(3,4-dichloro-phenyl)-2-[2-(4-methyl-piperazin-1-yl)benzylidene]-thiomorpholin-3-one, after which administration, the desired metabolite is formed in the subject's body through metabolism. Moreover, the administration route and dosage of 4-(3,4-dichloro-phenyl)-2-[2-(4-methyl-piperazin-1-yl)-benzylidene]-thiomorpholin-3-one can be varied, as desired, to obtain desired in vivo concentrations and rates of production of a metabolite.

When used for the treatment of one or more of the above conditions, 4-(3,4-dichloro-phenyl)2-[2-(4-methyl-piperazin-1-yl)-benzylidene]-thiomorpholin-3-one metabolites can be used (either co-administered separately or within the same pharmaceutical composition) in combination with one or more other agents as described hereinabove.

The pharmaceutically acceptable acid addition salts of the compounds of this invention can be formed of the compound itself, or of any of its esters, and include the pharmaceutically acceptable salts which are often used in pharmaceutical chemistry. For example, salts can be formed with inorganic or organic acids such as hydrochloric acid, hydrobromic acid, hydroiodic acid, sulfonic acids including such agents as naphthalenesulfonic, methanesulfonic and toluenesulfonic acids, sulfuric acid, nitric acid, phosphoric acid, tartaric acid, pyrosulfuric acid, metaphosphoric acid, succinic acid, formic acid, phthalic acid, lactic acid and the like, most preferable with hydrochloric acid, citric acid, benzoic acid, maleic acid, acetic acid and propionic acid.

The compounds of this invention, as discussed above, can be administered in the form of pharmaceutically acceptable salts. The salts are conveniently formed, as is usual in organic chemistry, by reacting a compound of this invention, when basic, with a suitable acid, such as have been described above. The salts are quickly formed in high yields at moderate temperatures, and often are prepared by merely isolating the compound from a suitable acidic wash as the final step of the synthesis. The salt-forming acid is dissolved in an appropriate organic solvent, or aqueous organic solvent, such as an alkanol, ketone or ester. On the other hand, if a compound of this invention is desired in the free base form, it is isolated from a basic final wash step, according to the usual practice. A preferred technique for preparing hydrochlorides is to dissolve the free base in a suitable solvent and dry the solution thoroughly, as over molecular sieves, before bubbling hydrogen chloride gas through it.

When used as a medicament, the dose, of a compound of this invention to be administered to a human is rather widely variable and subject to the judgement of the attending physician. It should be noted that it can be desirable to adjust the dose of a compound when it is administered in the form of a salt, such as a laureate, the salt forming moiety of which has an appreciable molecular weight. The general range of effective administration rates of the compounds is from about mg/day to about mg/day. A preferred range is from about mg/day to about mg/day. Of course, it is often practical to administer the daily dose of compound in portions, at various hours of the day. However, in any given case, the amount of compound administered will depend on such factors as the solubility of the active component, the formulation used and the route of administration.

The route of administration of the compounds of this invention is not critical. The compounds can be absorbed from the alimentary tract, however, the compounds can be administered percutaneously, or as suppositories for absorption by the rectum, if desired in a given instance. All of the usual types of compositions can be used, including tablets, chewable tablets, capsules, solutions, parenteral solutions, troches, suppositories and suspensions. Compositions are formulated to contain a daily dose, or a convenient fraction of daily dose, in a dosage unit, which can be a single tablet or capsule or convenient volume of a liquid.

In general, all of the compositions are prepared according to methods typically in pharmaceutical chemistry and/or isolated from in vivo or in vitro metabolism reactions such as those exemplified herein. The parent compound, 4-(3,4-dichloro-phenyl)-2-[2-(4-methyl-piperazin-1-yl)-benzylidene]-thiomorpholin-3-one, is prepared by those procedures outlined and/or exemplified in U.S. Pat. No. 6,380,186. The metabolites can be synthesized directly or can be formed by in vitro or in vivo enzymatic or metabolic reactions such as those described in the Examples.

Methods of formulation are well known in the art and are disclosed, for example, in Remington: The Science and Practice of Pharmacy, Mack Publishing Company, Easton, Pa., 19th Edition (1995). Pharmaceutical compositions for use within the present invention can be in the form of sterile, non-pyrogenic liquid solutions or suspensions, coated capsules, suppositories, lyophilized powders, transdermal patches or other forms known in the art.

Capsules are prepared by mixing the compound with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets are prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant can be added in a tablet formulation to prevent the tablet and punches from sticking in the die. The lubricant is chosen from such slippery solids as talc, magnesium and calcium stearate, stearic acid and hydrogenated vegetable oils.

Tablet disintegrators are substances which facilitate the disintegration of a tablet to release a compound when the tablet becomes wet. They include starches, clays, celluloses, algins and gums, more particularly, corn and potato starches, methylcellulose, agar, bentonite, wood cellulose, powdered natural sponge, cation-exchange resins, alginic acid, guar gum, citrus pulp and carboxymethylcellulose, for example, can be used as well as sodium lauryl sulfate.

Tablets are often coated with sugar as a flavor and sealant, or with film-forming protecting agents to modify the dissolution properties of the tablet. The compounds can also be formulated as chewable tablets, by using large amounts of pleasant-tasting substances such as mannitol in the formulation, as is now well-established in the art.

When it is desired to administer a compound as a suppository, the typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compounds can be delayed or prolonged by proper formulation. For example, a slowly soluble pellet of the compound can be prepared and incorporated in a tablet or capsule. The technique can be improved by making pellets of several different dissolution rates and filling capsules with a mixture of the pellets. Tablets or capsules can be coated with a film which resists dissolution for a predictable period of time. Even the parenteral preparations can be made long-acting, by dissolving or suspending the compound in oily or emulsified vehicles which allow it to disperse only slowly in the serum.

Unless otherwise indicated:

“Treating” refers to, and includes, reversing, alleviating, inhibiting the progress of, or preventing, a disease, disorder or condition, or one or more symptoms thereof; and, “treatment” and “therapeutically” refer to the act of treating, as defined above.

“Enhanced serotonergic neurotransmission,” as used herein, refers to increasing or improving the neuronal process whereby serotonin is released by a pre-synaptic cell upon excitation and crosses the synapse to stimulate or inhibit the post-synaptic cell.

“Chemical dependency,” as used herein, means an abnormal craving or desire for, or an addiction to a drug. Such drugs are generally administered to the affected individual by any of a variety of means of administration, including oral, parenteral, nasal or by inhalation. Examples of chemical dependencies treatable by the methods of the present invention are dependencies on alcohol, nicotine, cocaine, heroin, phenobarbital, and benzodiazepines (e.g., Valium (trademark)). “Treating a chemical dependency,” as used herein, means reducing or alleviating such dependency.

“Subject” is an animal, including mammals, and including human beings.

“Gluc.” refers to a glucoronide substituent. Glucuronic acid reacts with an acid or alcohol or phenol moiety on the metabolite or parent compound to form the “glucuride.” Glucuronic acid is the substituent that is transferred to a metabolite from the phase II conjunction reaction of glucuronidation.

“Purified and isolated” includes susbtantially pure and isolated sufficient for purposes of the invention as understood by the artisan.

The invention includes isotopically-labeled compounds identical to those of Formulae (I) to (XXIV) and other compounds of the invention save for one or more atoms being replaced by one of atomic mass or mass number different from that usually found in nature as understood by the artisan.

“Co-administration” of a combination of a 4-(3,4-dichloro-phenyl)-2-[2-(4-methyl-piperazin-1-yl)-benzylidene]-thiomorpholin-3-one metabolite and an additional compound or additional compounds means that these components can be administered together as a composition or as part of the same, unitary dosage form. “Co-administration” also includes administering a 4-(3,4-dichloro-phenyl)-2-[2-(4-methyl-piperazin-1-yl)-benzylidene]-thiomorpholin-3-one metabolite and an additional compound or additional compounds separately but as part of the same therapeutic treatment program or regimen. The components need not necessarily be administered at essentially the same time, although they can if so desired. Thus “co-administration” includes, for example, administering a 4-(3,4-dichloro-phenyl)-2-[2-(4-methyl-piperazin-1-yl)benzylidene]-thiomorpholin-3-one metabolite and an additional compound as separate dosages or dosage forms, but at the same time. “Co-administration” also includes separate administration at different times and in any order. For example, where appropriate a patient can take one or more component(s) of the treatment in the morning and the one or more of the other component(s) at night.

The term “prodrug” means compounds that are transformed in vivo to yield a compound of the present invention. The transformation can occur by various mechanisms, such as through hydrolysis in blood. A good discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of the present invention contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-((C₁-C₂))alkylamino(C₂-C₃)alkyl (such as p-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₁-C₃)alkyl.

Similarly, if a compound of the present invention comprises an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanoyl, arylacyl and α-aminoacyl, or α-aminoacylα-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

If a compound of the present invention comprises an amine functional group, a prodrug can be formed by the replacement of a hydrogen atom in the amine group with a group such as R^{X}-carbonyl, R^{X} O-carbonyl, N R^{X} R^{X1}-carbonyl where R^{X} and R^{X1} are each independently ((C₁-C₁₀)alkyl, (C₃-C₇)cycloalkyl, benzyl, or R^{x}-carbonyl is a natural α-aminoacyl or natural α-aminoacyl-natural α-aminoacyl, —C(OH)C(O)OY^{x} wherein (Y^{x} is H, (C₁-C₆)alkyl or benzyl), C(OY^{x0}) Y^{x} wherein Y^{x0} is (C₁-C₄)alkyl and Y.sup.x1 is ((C₁-C₆)alkyl, carboxy(C₁-C₆)alkyl, amino(C₁-C₄)alkyl or mono-N- or di-N,N-(C₁-C₆)alkylaminoalkyl, —C(Y^{x2}) Y^{x3} wherein Y^{x2} is H or methyl and Y^{x3} is mono-N- or di-N,N-(C₁-C₆)alkylamino, morpholino, piperidin-1-yl or pyrrolidin-1-yl.

As used herein, the term “effective amount” means an amount of compound of the methods of the present invention that is capable of treating the specific diseases and pathological conditions. The specific dose of a compound administered according to this invention will, of course, be determined by the particular circumstances surrounding the case including, for example, the compound administered, the route of administration, the state of being of the subject, and the severity of the pathological condition being treated.

Advantageously, the present invention also provides kits for use by a consumer for treating disease. The kits comprise a) a pharmaceutical composition comprising an estrogen agonist/antagonist and a pharmaceutically acceptable carrier, vehicle or diluent; and, optionally, b) instructions describing a method of using the pharmaceutical composition for treating the specific disease. The instructions can also indicate that the kit is for treating disease while substantially reducing the concomitant liability of adverse effects associated with estrogen administration.

A “kit” as used in the instant application includes a container for containing the separate unit dosage forms such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets can be contained in a bottle which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or can have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It can be desirable to provide a written memory aid, where the written memory aid is of the type containing information and/or instructions for the physician, pharmacist or subject, e.g., in the form of numbers next to the tablets or capsules whereby the numbers correspond with the days of the regimen which the tablets or capsules so specified should be ingested or a card which contains the same type of information. Another example of such a memory aid is a calendar printed on the card e.g., as follows “First Week, Monday, Tuesday,” . . . etc . . . . “Second Week, Monday, Tuesday, . . . ” etc. Other variations of memory aids will be readily apparent. A “daily dose” can be a single tablet or capsule or several tablets or capsules to be taken on a given day.

Another specific embodiment of a kit is a dispenser designed to dispense the daily doses one at a time. Preferably, the dispenser is equipped with a memory-aid, so as to further facilitate compliance with the regimen. An example of such a memory-aid is a mechanical counter, which indicates the number of daily doses that, has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The metabolites of formula I that are basic in nature are capable of forming a wide variety of different salts with various inorganic and organic acids. Although such salts must be pharmaceutically acceptable for administration to animals, it is often desirable in practice to initially isolate a compound of the formula I from the reaction mixture as a pharmaceutically unacceptable salt and then simply convert the latter back to the free base compound by treatment with an alkaline reagent, and subsequently convert the free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid addition salts of the base compounds of this invention are those which form non-toxic acid addition salts, i.e., salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate (i.e., 1,1′-methylene-bis-(2-hydroxy-3-naphthoate)) salts.

Those metabolites of formula I which are also acidic in nature, e.g., where R³ includes a COOH or tetrazole moiety, are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the herein described acidic metabolites of formula I. These non-toxic base salts include those derived from such pharmacologically acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they can also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum product yields.

Metabolites of formula I and their pharmaceutically acceptable salts (hereinafter also referred to, collectively, as “the active compounds”) are useful psychotherapeutics and are potent agonists and/or antagonists of the serotonin 1A (5-HT_{1A}) and/or serotonin 1D (5-HT_{1D}) receptors. The active compounds are useful in the treatment of hypertension, all forms of depression (e.g., depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition), bipolar disorder (including in the depressed phase), generalized anxiety disorder, social anxiety, separation anxiety disorder, phobias (e.g., agoraphobia, social phobia and simple phobias), posttraumatic stress syndrome, avoidant personality disorder, premature ejaculation, eating disorders (e.g., binge eating disorder, anorexia nervosa and bulimia nervosa), obesity, chemical dependencies (e.g., addictions to alcohol, cocaine, heroin, phenobarbital, marijuana, nicotine and benzodiazepines), duster headache, migraine, pain, Alzheimer's disease, obsessive-compulsive disorder; panic disorder with and without agoraphobia; memory disorders (e.g., dementia, amnestic disorders, and age-related cognitive decline (ARCD)), Parkinson's diseases (e.g., dementia in Parkinson's disease, neuroleptic-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania, headache (associated with vascular disorders) autism, pervasive developmental disorder NOS, Asperger's disorder, selective mutism, chronic motor or vocal tic disorder, somatization disorder, insomnia, intermittent explosive disorder, pyromania, pathological gambling, impulse-control disorder, premenstrual dysphoric disorder, and attention-deficit/hyperactivity disorder (ADHD).

The affinities of the compounds of this invention for the various serotonin-1 receptors can be determined using standard radioligand binding assays as described in the literature. The 5-HT_{1A} affinity can be measured using the procedure of Hoyer et al. (Brain Res., 376, 85 (1986)). The 5-HT_{1D} affinity can be measured using the procedure of Heuring and Peroutka (J. Neurosci., 7, 894 (1987)).

The in vitro activity of the compounds of the present invention at the 5-HT_{1D} binding site can be determined according to the following procedure. Bovine caudate tissue is homogenized and suspended in 20 volumes of a buffer containing 50 mM TRIS-hydrochloride (tris[hydroxymethyl]aminomethane hydrochloride) at a pH of 7.7. The homogenate is then centrifuged at 45,000 G for 10 minutes. The supernatant is then discarded and the resulting pellet resuspended in approximately 20 volumes of 50 mM TRIS.hydrochloride buffer at pH 7.7. This suspension is then pre-incubated for 15 minutes at 37° C., after which the suspension is centrifuged again at 45,000 G for 10 minutes and the supernatant discarded. The resulting pellet (approximately 1 gram) is resuspended in 150 ml of a buffer of 15 mM TRIS.hydrochloride containing 0.01 percent ascorbic acid with a final pH of 7.7 and also containing 10 μM pargyline and 4 mM calcium chloride (CaCl₂). The suspension is kept on ice at least 30 minutes prior to use.

The inhibitor, control or vehicle is then incubated according to the following procedure. To 50 μl of a 20 percent dimethylsulfoxide (DMSO)/80 percent distilled water solution is added 200 μl of tritiated 5-hydroxytryptamine (2 nM) in a buffer of 50 mM TRIS□hydrochloride containing 0.01 percent ascorbic acid at pH 7.7 and also containing 10 □M pargyline and 4 μM calcium chloride, plus 100 nM of 8-hydroxy-DPAT (dipropylaminotetraline) and 100 nM of mesulergine. To this mixture is added 750 μl of bovine caudate tissue, and the resulting suspension is vortexed to ensure a homogenous suspension. The suspension is then incubated in a shaking water bath for 30 minutes at 25° C. After incubation is complete, the suspension is filtered using glass fiber filters (e.g., Whatman GF/B-filters™). The pellet is then washed three times with 4 ml of a buffer of 50 mM TRIS.hydrochloride at pH 7.7. The pellet is then placed in a scintillation vial with 5 ml of scintillation fluid (aquasol 2™) and allowed to sit overnight. The percent inhibition can be calculated for each dose of the compound. An IC₅₀ value can then be calculated from the percent inhibition values.

The activity of the compounds of the present invention for 5-HT_{1A} binding ability can be determined according to the following procedure. Rat brain cortex tissue is homogenized and divided into samples of 1 gram lots and diluted with 10 volumes of 0.32 M sucrose solution. The suspension is then centrifuged at 900 G for 10 minutes and the supernate separated and recentrifuged at 70,000 G for 15 minutes. The supernate is discarded and the pellet re-suspended in 10 volumes of 15 mM TRIS.hydrochloride at pH 7.5. The suspension is allowed to incubate for 15 minutes at 37° C. After pre-incubation is complete, the suspension is centrifuged at 70,000 G for 15 minutes and the supernate discarded. The resulting tissue pellet is resuspended in a buffer of 50 mM TRIS□hydrochloride at pH 7.7 containing 4 mM of calcium chloride and 0.01 percent ascorbic acid. The tissue is stored at −70□C until ready for an experiment. The tissue can be thawed immediately prior to use, diluted with 10 μm pargyline and kept on ice.

The tissue is then incubated according to the following procedure. Fifty microliters of control, inhibitor, or vehicle (1 percent DMSO final concentration) is prepared at various dosages. To this solution is added 200 μl of tritiated DPAT at a concentration of 1.5 nM in a buffer of 50 mM TRIS.hydrochloride at pH 7.7 containing 4 mM calcium chloride, 0.01 percent ascorbic acid and pargyline. To this solution is then added 750 μl of tissue and the resulting suspension is vortexed to ensure homogeneity. The suspension is then incubated in a shaking water bath for 30 minutes at 37° C. The solution is then filtered, washed twice with 4 ml of 10 mM TRIS.hydrochloride at pH 7.5 containing 154 mM of sodium chloride. The percent inhibition is calculated for each dose of the compound, control or vehicle. IC₅₀ values are calculated from the percent inhibition values.

The agonist and antagonist activities of the compounds of the invention at 5-HT_{1A} and 5-HT_{1D} receptors can be determined using a single saturating concentration according to the following procedure. Male Hartley guinea pigs are decapitated and 5-HT_{1A} receptors are dissected out of the hippocampus, while 5-HT_{1D} receptors are obtained by slicing at 350 mM on a McIlwain tissue chopper and dissecting out the substantia nigra from the appropriate slices. The individual tissues are homogenized in 5 mM HEPES buffer containing 1 mM EGTA (pH 7.5) using a hand-held glass-Teflon® homogenizer and centrifuged at 35,000×g for 10 minutes at 4° C. The pellets are resuspended in 100 mM HEPES buffer containing 1 mM EGTA (pH 7.5) to a final protein concentration of 20 mg (hippocampus) or 5 mg (substantia nigra) of protein per tube. The following agents are added so that the reaction mix in each tube contained 2.0 mM MgCl₂, 0.5 mM ATP, 1.0 mM CAMP, 0.5 mM IBMX, 10 mM phosphocreatine, 0.31 mg/mL creatine phosphokinase, 100 □M GTP and 0.5-1 microcuries of [³²P]-ATP (30 Ci/mmol: NEG-003—New England Nuclear). Incubation is initiated by the addition of tissue to siliconized microfuge tubes (in triplicate) at 30° C. for 15 minutes. Each tube receives 20 μL tissue, 10 μL drug or buffer (at 10× final concentration), 10 μL 32 nM agonist or buffer (at 10× final concentration), 20 μL forskolin (3 □M final concentration) and 40 □L of the preceding reaction mix. Incubation is terminated by the addition of 100 μL 2% SDS, 1.3 mM CAMP, 45 mM ATP solution containing 40,000 dpm [³H]-cAMP (30 Ci/mmol: NET-275—New England Nuclear) to monitor the recovery of CAMP from the columns. The separation of [³²P]-ATP and [P]-cAMP is accomplished using the method of Salomon et al., Analytical Biochemistry, 1974, 58, 541-548. Radioactivity is quantified by liquid scintillation counting. Maximal inhibition is defined by 10 □μM (R)8—OH-DPAT for 5-HT_{1A} receptors, and 320 nM 5-HT for 5-HT_{1D} receptors. Percent inhibitions by the test compounds are then calculated in relation to the inhibitory effect of (R)-8-OH-DPAT for 5-HT_{1A} receptors or 5-HT for 5-HT_{1D} receptors. The reversal of agonist induced inhibition of forskolin-stimulated adenylate cyclase activity is calculated in relation to the 32 nM agonist effect.

The compounds of the invention can be tested for in vivo activity for antagonism of 5-HT_{1D} agonist-induced hypothermia in guinea pigs according to the following procedure.

Male Hartley guinea pigs from Charles River, weighing 250-275 grams on arrival and 300-600 grams at testing, serve as subjects in the experiment. The guinea pigs are housed under standard laboratory conditions on a 7 a.m. to 7 p.m. lighting schedule for at least seven days prior to experimentation. Food and water are available ad libitum until the time of testing.

The compounds of the invention can be administered as solutions in a volume of 1 ml/kg. The vehicle used is varied depending on compound solubility. Test compounds are typically administered either sixty minutes orally (p.o.) or 0 minutes subcutaneously (s.c.) prior to a 5-HT_{1D} agonist, such as [3-(1-methylpyrrolidin-2-ylmethyl)-1H-indol-5-yl]-(3-nitropyridin-3-yl)-amine, which can be prepared as described in PCT publication WO93/11106, published Jun. 10, 1993 which is administered at a dose of 5.6 mg/kg, s.c. Before a first temperature reading is taken, each guinea pig is placed in a clear plastic shoe box containing wood chips and a metal grid floor and allowed to acclimate to the surroundings for 30 minutes. Animals are then returned to the same shoe box after each temperature reading. Prior to each temperature measurement each animal is firmly held with one hand for a 30-second period. A digital thermometer with a small animal probe is used for temperature measurements. The probe is made of semi-flexible nylon with an epoxy tip. The temperature probe is inserted 6 cm. into the rectum and held there for 30 seconds or until a stable recording is obtained. Temperatures are then recorded.

In p.o. screening experiments, a “pre-drug” baseline temperature reading is made at −90 minutes, the test compound is given at −60 minutes and an additional −30 minute reading is taken. The 5-HT_{1D} agonist is then administered at 0 minutes and temperatures are taken 30, 60, 120 and 240 minutes later.

In subcutaneous screening experiments, a pre-drug baseline temperature reading is made at −30 minutes. The test compound and 5-HT_{1D} agonists are given concurrently and temperatures are taken at 30, 60, 120 and 240 minutes later.

Data are analyzed with two-way analysis of variants with repeated measures in Newman-Keuls post hoc analysis.

The active compounds of the invention can be evaluated as anti-migraine agents by testing the extent to which they mimic sumatriptan in contracting the dog isolated saphenous vein strip (P. P. A. Humphrey et al., Br. J. Pharmacol., 94, 1128 (1988)). This effect can be blocked by methiothepin, a known serotonin antagonist. Sumatriptan is known to be useful in the treatment of migraine and produces a selective increase in carotid vascular resistance in the anesthetized dog. The pharmacological basis of sumatriptan efficacy has been discussed in W. Fenwick et al., Br. J. Pharmacol., 96, 83 (1989).

The serotonin 5-HT₁ agonist activity can be determined by the in vitro receptor binding assays, as described for the 5-HT_{1A} receptor using rat cortex as the receptor source and [³H]-8-OH-DPAT as the radioligand (D. Hoyer et al. Eur. J. Pharm., 118, 13 (1985)) and as described for the 5-HT_{1D} receptor using bovine caudate as the receptor source and [³H]serotonin as the radioligand (R. E. Heuring and S. J. Peroutka, J. Neuroscience, 7, 894 (1987)). Of the active compounds tested, all exhibited an IC₅₀ in either assay of 1 mM or less.

The metabolites of formula I can advantageously be used in conjunction with one or more other therapeutic agents, for instance, different antidepressant agents such as tricyclic antidepressants (e.g., amitriptyline, dothiepin, doxepin, trimipramine, butripyline, clomipramine, desipramine, imipramine, iprindole, lofepramine, nortriptyline or protriptyline), monoamine oxidase inhibitors (e.g., isocarboxazid, pheneizine or tranylcyclopramine) or 5-HT re-uptake inhibitors (e.g., fluvoxamine, sertraline, fluoxetine or paroxetine), and/or with antiparkinsonian agents such as dopaminergic antiparkinsonian agents (e.g., levodopa, preferably in combination with a peripheral decarboxylase inhibitor e.g., benserazide or carbidopa, or with a dopamine agonist e.g., bromocriptine, lysuride or pergolide). It is to be understood that the present invention covers the use of a metabolite of general formula (I) or a physiologically acceptable salt or solvate thereof in combination with one or more other therapeutic agents.

Metabolites of formula I and the pharmaceutically acceptable salts thereof, in combination with a 5-HT re-uptake inhibitor (e.g., fluvoxamine, sertraline, fluoxetine or paroxetine), preferably sertraline, or a pharmaceutically acceptable salt or polymorph thereof (the combination of a metabolite of formula I with a 5-HT re-uptake inhibitor is referred herein to as “the active combination”), are useful psychotherapeutics and can be used in the treatment of disorders the treatment of which is facilitated by enhanced serotonergic neurotransmission (e.g., hypertension, all forms of depression (e.g., depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition), bipolar disorder (including in the depressed phase), generalized anxiety disorder, social anxiety, separation anxiety disorder, phobias (e.g., agoraphobia, social phobia and simple phobias), posttraumatic stress syndrome, avoidant personality disorder, premature ejaculation, eating disorders (e.g., binge eating disorder, anorexia nervosa and bulimia nervosa), obesity, chemical dependencies (e.g., addictions to alcohol, cocaine, heroin, phenobarbital, marijuana, nicotine and benzodiazepines), cluster headache, migraine, pain, Alzheimer's disease, obsessive-compulsive disorder; panic disorder with and without agoraphobia; memory disorders (e.g., dementia, amnestic disorders, and age-related cognitive decline (ARCD)), Parkinson's diseases (e.g., dementia in Parkinson's disease, neuroleptic-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania, headache (associated with vascular disorders) autism, pervasive developmental disorder NOS, Asperger's disorder, selective mutism, chronic motor or vocal tic disorder, somatization disorder, insomnia, intermittent explosive disorder, pyromania, pathological gambling, impulse-control disorder, premenstrual dysphoric disorder, and attention-deficit/hyperactivity disorder (ADHD)).

Serotonin (5-HT) re-uptake inhibitors, preferably sertraline, exhibit positive activity against depression; chemical dependencies; anxiety disorders including panic disorder, generalized anxiety disorder, agoraphobia, simple phobias, social phobia, and post-traumatic stress disorder; obsessive-compulsive disorder; avoidant personality disorder and premature ejaculation in mammals, including humans, due in part to their ability to block the synaptosomal uptake of serotonin.

U.S. Pat. No. 4,536,518 describes the synthesis, pharmaceutical composition and use of sertraline for depression and is hereby incorporated by reference in its entirety.

Activity of the active combination as antidepressants and related pharmacological properties can be determined by methods (1)-(4) below, which are described in Koe, B. et al., Journal of Pharmacology and Experimental Therapeutics, 226 (3), 686-700 (1983). Specifically, activity can be determined by studying (1) their ability to affect the efforts of mice to escape from a swim-tank (Porsolt mouse “behavior despair” test), (2) their ability to potentiate 5-hydroxytryptophan-induced behavioral symptoms in mice in vivo, (3) their ability to antagonize the serotonin-depleting activity of p-chloroamphetamine hydrochloride in rat brain in vivo, and (4) their ability to block the uptake of serotonin, norepinephrine and dopamine by synaptosomal rat brain cells in vitro. The ability of the active combination to counteract reserpine hypothermia in mice in vivo can be determined according to the methods described in U.S. Pat. No. 4,029,731.

The compositions of the present invention can be formulated in a conventional manner using one or more pharmaceutically acceptable carriers. Thus, the active compounds of the invention can be formulated for oral, buccal, intranasal, parenteral (e.g., intravenous, intramuscular or subcutaneous) or rectal administration or in a form suitable for administration by inhalation or insufflation.

For oral administration, the pharmaceutical compositions can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition can take the form of tablets or lozenges formulated in conventional manner.

The active compounds of the invention can be formulated for parenteral administration by injection, including using conventional catheterization techniques or infusion. Formulations for injection can be presented in unit dosage form, e.g., in ampules or in multi-dose containers, with an added preservative. The compositions can take such forms as suspensions, solutions or emulsions in oily or aqueous vehicles, and can contain formulating agents such as suspending, stabilizing and/or dispersing agents. Alternatively, the active ingredient can be in powder form for reconstitution with a suitable vehicle, e.g., sterile pyrogen-free water, before use.

The active compounds of the invention can also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. The pressurized container or nebulizer can contain a solution or suspension of the active compound. Capsules and cartridges (made, for example, from gelatin) for use in an inhaler or insufflator can be formulated containing a powder mix of a compound of the invention and a suitable powder base such as lactose or starch.

A proposed dose of the active compounds of the invention for oral, parenteral or buccal administration to the average adult human for the treatment of the conditions referred to above (e.g., depression) is 0.1 to 200 mg of the active ingredient per unit dose which could be administered, for example, 1 to 4 times per day.

Aerosol formulations for treatment of the conditions referred to above (e.g., migraine) in the average adult human are preferably arranged so that each metered dose or “puff” of aerosol contains 20 μg to 1000 μg of the compound of the invention. The overall daily dose with an aerosol will be within the range 100 μg to 10 mg. Administration can be several times daily, for example 2, 3, 4 or 8 times, giving for example, 1, 2 or 3 doses each time.

In connection with the use of an active compound of this invention with a 5-HT re-uptake inhibitor, preferably sertraline, for the treatment of subjects possessing any of the above conditions, it is to be noted that these compounds can be administered either alone or in combination with pharmaceutically acceptable carriers by either of the routes previously indicated, and that such administration can be carried out in both single and multiple dosages. More particularly, the active combination can be administered in a wide variety of different dosage forms, i.e., they can be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the metabolites of formula I are present in such dosage forms at concentration levels ranging from about 0.5% to about 90% by weight of the total composition, i.e., in amounts which are sufficient to provide the desired unit dosage and a 5-HT re-uptake inhibitor, preferably sertraline, is present in such dosage forms at concentration levels ranging from about 0.5% to about 90% by weight of the total composition, i.e., in amounts which are sufficient to provide the desired unit dosage.

A proposed daily dose of an active compound of this invention in the combination formulation (a formulation containing an active compound of this invention and a 5-HT re-uptake inhibitor) for oral, parenteral, rectal or buccal administration to the average adult human for the treatment of the conditions referred to above is from about 0.01 mg to about 2000 mg, preferably from about 0.1 mg to about 200 mg of the active ingredient of formula I per unit dose which could be administered, for example, 1 to 4 times per day.

A proposed daily dose of a 5-HT re-uptake inhibitor, preferably sertraline, in the combination formulation for oral, parenteral or buccal administration to the average adult human for the treatment of the conditions referred to above is from about 0.1 mg to about 2000 mg, preferably from about 1 mg to about 200 mg of the 5-HT re-uptake inhibitor per unit dose which could be administered, for example, 1 to 4 times per day.

A preferred dose ratio of sertraline to an active compound of this invention in the combination formulation for oral, parenteral or buccal administration to the average adult human for the treatment of the conditions referred to above is from about 0.00005 to about 20,000, preferably from about 0.25 to about 2,000.

Aerosol combination formulations for treatment of the conditions referred to above in the average adult human are preferably arranged so that each metered dose or “puff” of aerosol contains from about 0.01 μg to about 100 mg of the active compound of this invention, preferably from about 1 μg to about 10 mg of such compound. Administration can be several times daily, for example 2, 3, 4 or 8 times, giving for example, 1, 2 or 3 doses each time.

Aerosol formulations for treatment of the conditions referred to above in the average adult human are preferably arranged so that each metered dose or “puff” of aerosol contains from about 0.01 mg to about 2000 mg of a 5-HT re-uptake inhibitor, preferably sertraline, preferably from about 1 mg to about 200 mg of sertraline. Administration can be several times daily, for example 2, 3, 4 or 8 times, giving for example, 1, 2 or 3 doses each time.

As previously indicated, a 5-HT re-uptake inhibitor, preferably sertraline, in combination with metabolites of formula I are readily adapted to therapeutic use as antidepressant agents. In general, these antidepressant compositions containing a 5-HT re-uptake inhibitor, preferably sertraline, and a compound of formula I are normally administered in dosages ranging from about 0.01 mg to about 100 mg per kg of body weight per day of a 5-HT re-uptake inhibitor, preferably sertraline, preferably from about 0.1 mg. to about 10 mg per kg of body weight per day of sertraline; with from about 0.001 mg. to about 100 mg per kg of body weight per day of a compound of formula I, preferably from about 0.01 mg to about 10 mg per kg of body weight per day of a compound of formula I, although variations will necessarily occur depending upon the conditions of the subject being treated and the particular route of administration chosen.

All references and patents cited herein are incorporated by reference.

The following schemes and examples are offered in illustration of the present invention; they are not to constrain the scope of the same in any way.

### EXAMPLES

### Example 1

### Isolation of 4-(3,4-dichloro-phenyl)-2-[2-(4-methyl-piperazin-1-yl)-benzylidene]-thiomorpholin-3-one metabolites in humans

Six healthy male human subjects were administered a single 10 mg (free base) oral dose of 4-(3,4-dichloro-phenyl)-2-[2-(4-methyl-piperazin-1-yl)-benzylidene]-thiomorpholin-3-one containing 100 μCi of [¹⁴C]-4-(3,4-dichloro-phenyl)-2-[2-(4-methyl-piperazin-1-yl)-benzylidene]-thiomorpholin-3-one (Lot# 49,800-103-1, specific activity 15.66 mCi/mmol, radiochemical purity >99%).

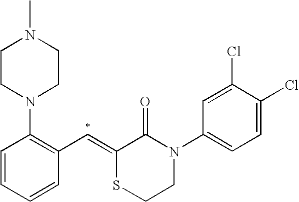

### 4-(3,4-DICHLORO-PHENYL)-2-[2-(4-METHYL-PIPERAZIN-1-YL)-BENZYLIDENE]THIOMOPHOLIN-3-ONE (*¹⁴C label)

a). Blood sufficient to provide approximately 10 mL plasma for 4-(3,4-dichloro-phenyl)-2-[2-(4-methyl-piperazin-1-yl)-benzylidene]-thiomorpholin-3-one metabolite identification was collected in heparinized tubes at the following approximate times: 2, 8, 24, 48, 96, and 192 hours after drug administration. Samples were centrifuged at approximately 4° C. and the plasma was stored in appropriately labeled, screw-capped opaque polypropylene tubes at −20° C. until analysis.

Plasma samples were pooled by human subject separately according to the method published by Raid, et al. (Raid, et al: Pharm. Res. (1991), 8(4), 541-543). Aliquots (1 mL) from each pool were extracted with 5 mL of acetonitrile. The mixtures were vortex-mixed for 3 minutes, sonicated for 30 minutes and then centrifuged for 7 minutes at 20° C. Aliquotes of the supernatant (2×500 μL) were counted to determine recovery. Approximately 95.1% of the radioactivity was recovered in the supernatants. The supernatants were combined according to subject number to two equal sets and dried in the Turbovap at room temperature. One set of the dried residue (six subjects) was reconstituted in 150 μL of 10 mM ammonium acetate (pH 4.0)/methanol/dimethyl sulfoxide (1.5:1.5:1) and aliquots of 100 μL were injected onto HPLC-Arc system for profiling. The other set of dried residues were combined and reconstituted in 300 μL of 10 mM ammonium acetate (pH 4.0)/methanol/dimethyl sulfoxide (1.5:1.5:1) and 90 μL was injected onto HPLC-MS/MS system for metabolite identification.

b). Urine samples were pooled according to sample volume. Approximately 2-5 mL of urine samples were evaporated under N₂ at room temperature in a Turbovap and reconstituted in 200 ul of 10 mM ammonium acetate (pH 4.0)/methanol/dimethyl sulfoxide (1.5:1.5:1). Aliquots of 100 μL were injected inot the HPLC-Arc system without further purification for profiling. Approximately 80 mL urine pools were dried in the Turbovap at room temperature and then reconstituted in 600 μL of 10 mM ammonium acetate (pH 4.0)/methanol/dimethyl sulfoxide (1.5:1.5:1). Aliquots of 80 μL were injected onto the HPLC-MS/MS system for metabolite identification.

c). Fecal samples were pooled according to sample weight. For metabolite profiling, approximately 1 g of each sample pool was extracted with 7 mL of acetonitrile/H₂O (6:1 v/v) twice, and 100 μL aliquots of both extractions were counted to determine recovery. Approximately 88.2% of the radioactivity was recovered in the supernatants. The supernatant was evaporated under N₂ in a Turbovap at room temperature and the residue was reconstituted at 500 μL of 10 mM ammonium acetate (pH 5.0)/methanol/dimethyl sulfoxide(1.5:1.5:1). Aliquots of 100 μL were injected onto the HPLC-Arc system for profiling. For metabolite identification, 10 g of each sample pool was extracted with 30 mL of Acetonitrile/H₂O (5:1 v/v) twice. The supernatants were combined and dried in Turbovap at room temperature. The dried residue was dissolved in 5 mL of water and extracted with 15 mL hexanes twice. The hexanes layer was removed and the aqueous layer was further extracted with 15 mL ethyl acetate twice. The extractions were combined and dried in the turbovap at room temperature and the residue was reconstituted in 360 μL of 10 mM ammonium acetate (pH 5.0)/methano/dimethyl sulfoxide (1.5:1.5:1). Aliquots of 90 μL were injected onto HPLC-MS/MS system for metabolite identification.

Based on the above methods of extracting metabolites of 4-(3,4-dichloro-phenyl)-2-[2-(4-methyl-piperazin-1-yl)-benzylidene]-thiomorpholin-3-one, the following data was determined.
Compound V m/z 448
Compound VII m/z 464
Compound VII m/z 434
Compound IX m/z 450
Compound X m/z 626
Compound XI m/z 480
Compound XII m/z 464
Compound XIII m/z 640
Compound XIV m/z 464
Compound XV m/z 640
Compound XVI m/z 464
Compound XVII m/z 444
Compound XX m/z 482
Compound XXI m/z 298
Compound XXII m/z 314
Compound XXIII m/z 299
Compound XXIV m/z 315

### Example 2

### Circulating Metabolite (M10A) Large Scale Biosynthesis of M10A

The reaction mixture consisted of 900 μL stock [¹⁴C]-4-(3,4-dichloro-phenyl)-2-[2-(4-methyl-piperazin-1-yl/benzylidene]-thiomorpholin-3-one (20 μM) (1 μg/μL in 50/50 MeOH/H₂O), 3600 μL rCYP 3A4 (37 nM) (9.6 mg protein/mL, 109 pmol P450/mg protein), 7550 μL rCYP 1A2 (25 nM) (8.6 mg protein/mL, 38.4 pmol P450/mg protein) and 77.95 mL of phosphate buffer (pH 7.4. The reaction was initiated with 10 mL cofactor solution (40 mg β-NADP, 160 mg D, L-isocitric acid +9.785 mL of 125 mM MgCl₂+215 μL of isocitric acid dehydrogenase). This gave a final incubation volume of 100 mL. The incubation was carried out in 37° C. for one hour.

For NMR analysis, same set of experiment was carried out using cold 4-(3,4-dichloro-phenyl)-2-[2-(4-methyl-piperazin-1-yl)-benzylidene]-thiomorpholin-3-one.

Following incubation, the reaction mixture was divided equally into 50 tubes (2.0 mL in each tube) and stopped by the addition of 5.0 mL MTBE to each tube. Each tube was vortex-mixed using a Multivotexer, centrifuge for 6 min, then flash freeze on methanol with dry ice. The supernatants were combined and dried under Turbovap.

To generate M10A, the large scale incubated sample was reduced by NaBH₄: The dried incubated sample was reconstituted in 5 mL anhydrous methanol, dried, then was reconstituted in 200 μL anhydrous methanol again, about 20 μg NaBH₄ (98%, Aldrich Chemical Company, Milwaukee, Mich., USA) was added. The reaction was carried on ice for 30 minutes and dried under Turbovap. The same experiment was carried for unlabelled 4-(3,4-dichloro-phenyl)-2-[2-(4-methyl-piperazin-1-yl)benzylidene]-thiomorpholin-3-one.

M10A co-eluted with M10 and had a retention time of ˜30.5 minutes on the LC-Arc system. M10A showed a protonated molecular ion at m/z 446.

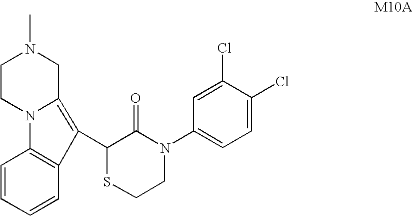

The following table shows the percentage of plasma metabolites of in humans after oral administration of the compound of formula I (of 4-(3,4-dichloro-phenyl)-2-[2-(4-methyl-piperazin-1-yl)-benzylidene]-thiomorpholin-3-one).

TABLE 5-3
Percentages of Plasma Metabolites of Formula I in Human
After Oral Administration of [¹⁴C]-Formula I

| | Retention | % of Total Radioactivity in Plasma | | | | | | |
|---|---|---|---|---|---|---|---|---|
| Metabolite | Time(min) | Subject#1 | Subject#2 | Subject#3 | Subject#4 | Subject#5 | Subject#6 | Mean |
| M8 | 27.9 | 16.4 | 25.0 | 22.6 | 15.3 | 23.1 | 19.1 | 20.2 |
| M9 | 27.9 | 4.0 | 2.2 | 4.5 | 5.2 | 2.7 | 4.4 | 3.8 |
| M10 | 30.5 | 4.4 | 3.6 | 5.1 | 7.3 | 4.6 | 8.2 | 5.5 |
| M10A | 30.5 | 60.6 | 57.6 | 62.8 | 72.2 | 69.6 | 68.3 | 65.2 |
| Total | | 85.4 | 88.4 | 95.0 | 100.0 | 100.0 | 100.0 | 94.8 |

Based on a reading of the present description and claims, certain modifications to the compositions and methods described herein will be apparent to one of ordinary skill in the art. The claims appended hereto are intended to encompass these modifications.

1. A purified and isolated metabolite of the compound of Formula (I): 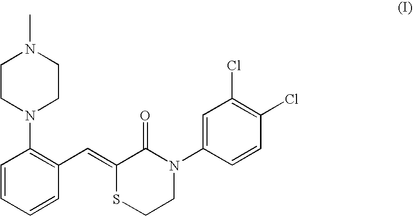
the racemic-diastereomeric mixtures and optical isomers thereof, the prodrugs thereof, and the pharmaceutiaclly acceptable salts of said metabolites, racemic-diastereomeris mixtures, optical isomers, and prodrugs.

2. An isolated and purifed metabolite of 4-(3,4-dichloro-phenyl)-2-[2-(4-methyl-piperazin-1-yl)
benzylidene]-thiomorpholin-3-one corresponding to Formula (II) or (III): 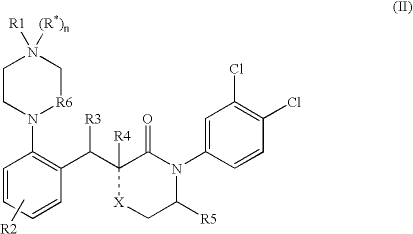
wherein
R1 is H or CH₃;
R2 is H, CH₃, or Gluc;
R3 is H or OH, or R3 and R4 form a bond, or R3 and R6 together with the C and N atoms to which they are respectively attached form a 5-membered unsaturated ring, or R3 and R4 together are —O— and form a 3-membered epoxide ring with the C atom to which they are respectively attached;
R4 is H, CH₃ when X is S, or R4 is (═O) when X is SH;
R5 is H, OH or (═O); and
R6 is —CH₂;
X is S when bond (—) is present, and SH when bond (—) is absent;
n is 0 or 1;
R* is O or is a bond between the N atom to which it is attached and an adjacent C atom whereby in either case said N atom has a positive charge; and
the racemic-diastereomeric mixtures and optical isomers thereof, the prodrugs thereof, and the pharmaceutically acceptable salts of said metabolites, racemic-diastereomer mixtures, optical isomers, and prodrugs;
with the proviso that R3 and R4 do not form a bond when R1 is CH₃, R6 is CH₂ and R2 and R3 are each hydrogen; or 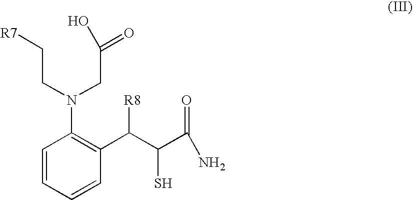
wherein
R7 is NH2 or OH; and
R8 is H or OH; and
the racemic-diastereomeric mixtures and optical isomers thereof, the prodrugs thereof, and the pharmaceutically acceptable salts of said metabolites, racemic-diastereomer mixtures, optical isomers, and prodrugs.

3. The metabolite of claim 1 selected from the group consisting of:

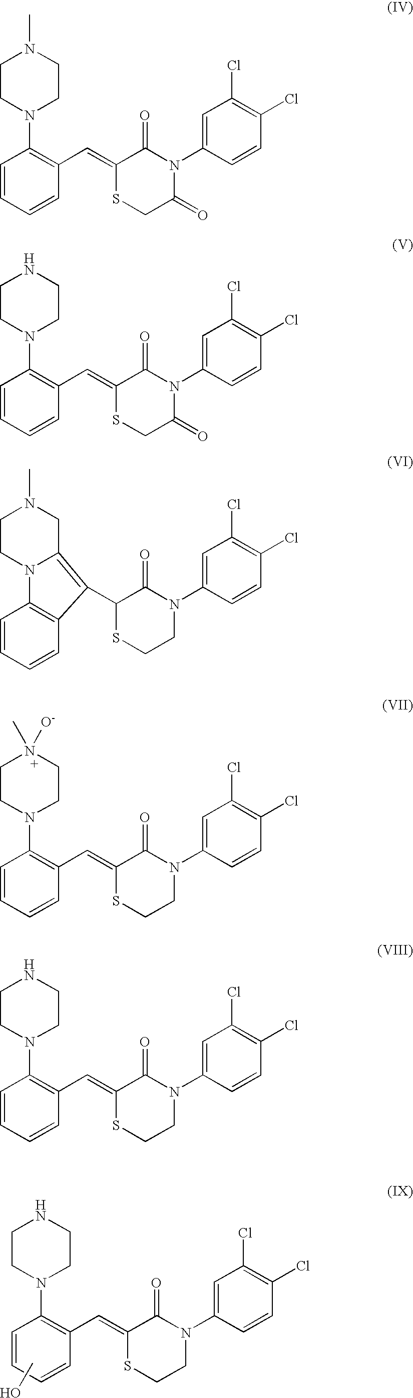

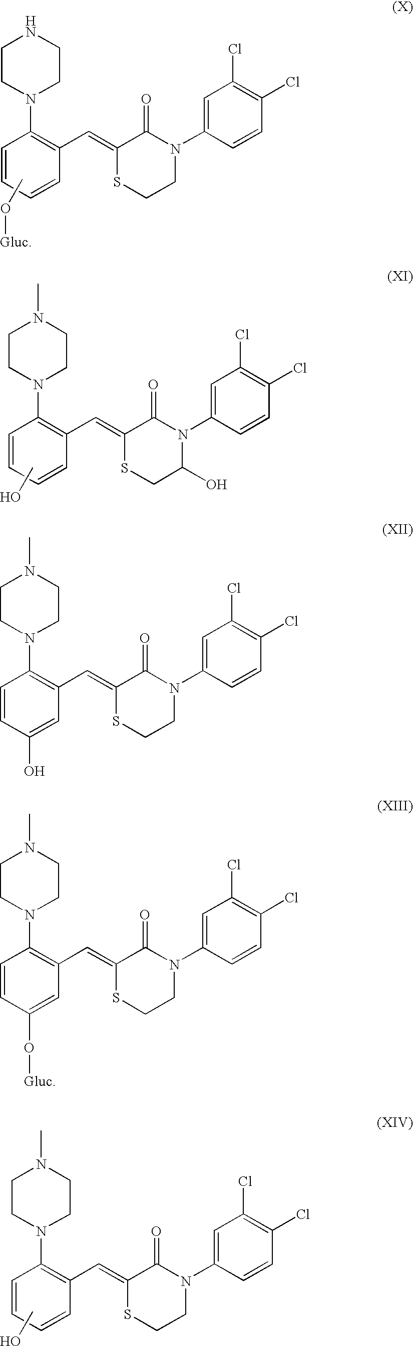

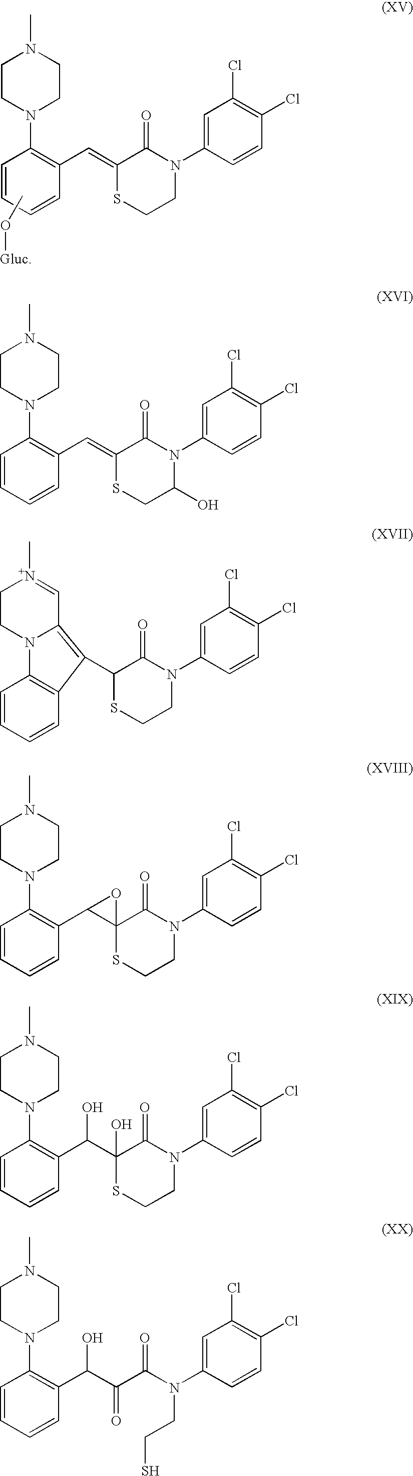

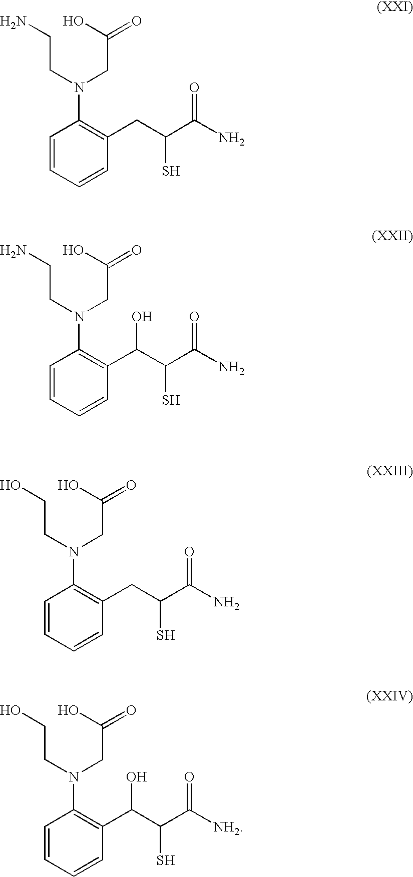

4. A pharmaceutical composition comprising one or more metabolites of claim 2 and a pharmaceutically acceptable carrier.

5. A pharmaceutical composition comprising one or more metabolites of claim 3 and a pharmaceutically acceptable carrier.

6. An assay for assessing the metabolic fate of 4-(3,4-dichloro-phenyl)-2-[2-(4-methyl-piperazin-1-yl)-benzylidene]-thiomorpholin-3-one, comprising the metabolite of claim 2.

7. A pharmaceutical composition comprising one or more metabolites according to claim 1 or a pharmaceutically acceptable salt thereof and a pharmaceutically acceptable carrier.

8. A method for treating a disorder or condition selected from hypertension, all forms of depression, depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition, bipolar disorder, bipolar disorder-depressed phase, generalized anxiety disorder, phobias, agoraphobia, social anxiety, social phobia, simple phobias, separation anxiety disorder, posttraumatic stress syndrome, avoidant personality disorder, premature ejaculation, eating disorders, binge eating disorder, anorexia nervosa, bulimia nervosa, obesity; chemical dependencies and addictions to alcohol, cocaine, heroin, phenobarbital, nicotine, marijuana and benzodiazepines; cluster headache, migraine, pain, Alzheimer's disease, obsessive-compulsive disorder, panic disorder, panic disorder with agoraphobia, memory disorders, dementia, amnestic disorders, and age-related cognitive decline (ARCD), Parkinson's diseases, dementia in Parkinson's disease, neuroleptic-induced parkinsonism and tardive dyskinesias, endocrine disorders, hyperprolactinaemia, vasospasm, vasospasm in the cerebral vasculature, cerebellar ataxia; gastrointestinal tract disorders involving changes in motility and secretion; negative symptoms of schizophrenia, premenstrual syndrome, fibromyalgia syndrome, stress incontinence, Tourette's syndrome, trichotillomania, kleptomania, male impotence, cancer, small cell lung carcinoma, chronic paroxysmal hemicrania, headache associated with vascular disorders, autism, pervasive developmental disorder NOS, Asperger's disorder, selective mutism, chronic motor or vocal tic disorder, somatization disorder, insomnia, intermittent explosive disorder, pyromania, pathological gambling, impulse-control disorder, premenstrual dysphoric disorder and attention-deficit/hyperactivity disorder (ADHD), in a mammal, comprising administering to a mammal in need of such treatment an amount of one or more metabolites according to claim 1, or a pharmaceutically acceptable salt thereof, that is effective in treating such disorder or condition.

9. A method for treating a disorder or condition selected from attention-deficit/hyperactivity disorder (ADHD), bipolar disorder, bipolar disorder-depressed phase; mild, moderate, or severe depression with or without atypical features, melancholic features, psychotic features, catatonic features; seasonal affective disorder, postpartum depression, geriatric depression, chronic depression, dysthymia, adjustment disorder with depressed mood, adjustment disorder with anxiety, depressed mood, mixed anxiety and depression, substance induced mood disorder, mood disorder secondary to a general medical condition, social anxiety, separation anxiety disorder, binge eating disorder, dependencies and addictions to marijuana, panic disorder with agoraphobia; autism, pervasive developmental disorder NOS, Asperger's disorder, selective mutism, chronic motor or vocal tic disorder, somatization disorder, insomnia, intermittent explosive disorder, pyromania, pathological gambling, impulse-control disorder, and premenstrual dysphoric disorder, in a mammal, comprising administering to a mammal in need of such treatment an amount of one or more metabolites according to claim 1, or a pharmaceutically acceptable salt thereof, that is effective in treating such disorder or condition.

10. A method for treating a disorder or condition that can be treated by enhancing serotonergic neurotransmission in a mammal, comprising administering to a mammal in need of such treatment an amount of one or more metabolites according to claim 1, or a pharmaceutically acceptable salt thereof, that is effective in treating such disorder or condition.

11. A method for treating a disorder or condition selected from hypertension, all forms of depression, depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition, bipolar disorder, bipolar disorder-depressed phase, generalized anxiety disorder, phobias, agoraphobia, social anxiety, social phobia, simple phobias, separation anxiety disorder, posttraumatic stress syndrome, avoidant personality disorder, premature ejaculation, eating disorders, binge eating disorder, anorexia nervosa, bulimia nervosa, obesity; chemical dependencies and addictions to alcohol, cocaine, heroin, phenobarbital, nicotine, marijuana and benzodiazepines; cluster headache, migraine, pain, Alzheimer's disease, obsessive-compulsive disorder, panic disorder, panic disorder with agoraphobia, memory disorders, dementia, amnestic disorders, and age-related cognitive decline (ARCD), Parkinson's diseases, dementia in Parkinson's disease, neuroleptic-induced parkinsonism and tardive dyskinesias, endocrine disorders, hyperprolactinaemia, vasospasm, vasospasm in the cerebral vasculature, cerebellar ataxia; gastrointestinal tract disorders involving changes in motility and secretion; negative symptoms of schizophrenia, premenstrual syndrome, fibromyalgia syndrome, stress incontinence, Tourette's syndrome, trichotillomania, kleptomania, male impotence, cancer, small cell lung carcinoma, chronic paroxysmal hemicrania, headache associated with vascular disorders, autism, pervasive developmental disorder NOS, Asperger's disorder, selective mutism, chronic motor or vocal tic disorder, somatization disorder, insomnia, intermittent explosive disorder, pyromania, pathological gambling, impulse-control disorder, premenstrual dysphoric disorder and attention-deficit/hyperactivity disorder (ADHD), in a mammal, comprising administering to a mammal requiring such treatment a serotonin receptor antagonizing or agonizing effective amount of one or more metabolites according to claim 1 or a pharmaceutically acceptable salt thereof.

12. A pharmaceutical composition for treating a disorder or condition that can be treated by enhancing serotonergic neurotransmission in a mammal, comprising:
a) a pharmaceutically acceptable carrier;
b) one or more metabolites according to claim 1; and
c) one or more 5-HT re-uptake inhibitors or a pharmaceutically acceptable salt thereof;
wherein the amount of the active compounds are such that the combination is effective in treating such disorder or condition.

13. A method for treating a disorder or condition that can be treated by enhancing serotonergic neurotransmission in a mammal, comprising administering to a mammal requiring such treatment:
a) one or more metabolites according to claim 1; and
b) one or more 5-HT re-uptake inhibitors or a pharmaceutically acceptable salt thereof;
wherein the amounts of the active compounds are such that the combination is effective in treating such disorder or condition.

14. A pharmaceutical composition according to claim 12, wherein the 5-HT re-uptake inhibitor is sertraline or a pharmaceutically acceptable salt thereof.

15. A method according to claim 13, wherein the 5-HT re-uptake inhibitor is sertraline or a pharmaceutically acceptable salt thereof.

16. A method for treating a disorder or condition selected from hypertension, all forms of depression, depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition, bipolar disorder, bipolar disorder-depressed phase, generalized anxiety disorder, phobias, agoraphobia, social anxiety, social phobia, simple phobias, separation anxiety disorder, posttraumatic stress syndrome, avoidant personality disorder, premature ejaculation, eating disorders, binge eating disorder, anorexia nervosa, bulimia nervosa, obesity; chemical dependencies and addictions to alcohol, cocaine, heroin, phenobarbital, nicotine, marijuana and benzodiazepines; cluster headache, migraine, pain, Alzheimer's disease, obsessive-compulsive disorder, panic disorder, panic disorder with agoraphobia, memory disorders, dementia, amnestic disorders, and age-related cognitive decline (ARCD), Parkinson's diseases, dementia in Parkinson's disease, neuroleptic-induced parkinsonism and tardive dyskinesias, endocrine disorders, hyperprolactinaemia, vasospasm, vasospasm in the cerebral vasculature, cerebellar ataxia; gastrointestinal tract disorders involving changes in motility and secretion; negative symptoms of schizophrenia, premenstrual syndrome, fibromyalgia syndrome, stress incontinence, Tourette's syndrome, trichotillomania, kleptomania, male impotence, cancer, small cell lung carcinoma, chronic paroxysmal hemicrania, headache associated with vascular disorders, autism, pervasive developmental disorder NOS, Asperger's disorder, selective mutism, chronic motor or vocal tic disorder, somatization disorder, insomnia, intermittent explosive disorder, pyromania, pathological gambling, impulse-control disorder, premenstrual dysphoric disorder and attention-deficit/hyperactivity disorder (ADHD), in a mammal, comprising administering to a mammal requiring such treatment:
a) a compound according to claim 1; and
b) a 5-HT re-uptake inhibitor or a pharmaceutically acceptable salt thereof;
wherein the amounts of the active compounds are such that the combination is effective in treating such disorder or condition.

17. A method for treating a disorder or condition selected from hypertension, all forms of depression, depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia;
mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition, bipolar disorder, bipolar disorder-depressed phase, generalized anxiety disorder, phobias, agoraphobia, social anxiety, social phobia, simple phobias, separation anxiety disorder, posttraumatic stress syndrome, avoidant personality disorder, premature ejaculation, eating disorders, binge eating disorder, anorexia nervosa, bulimia nervosa, obesity; chemical dependencies and addictions to alcohol, cocaine, heroin, phenobarbital, nicotine, marijuana and benzodiazepines; cluster headache, migraine, pain, Alzheimer's disease, obsessive-compulsive disorder, panic disorder, panic disorder with agoraphobia, memory disorders, dementia, amnestic disorders, and age-related cognitive decline (ARCD), Parkinson's diseases, dementia in Parkinson's disease, neuroleptic-induced parkinsonism and tardive dyskinesias, endocrine disorders, hyperprolactinaemia, vasospasm, vasospasm in the cerebral vasculature, cerebellar ataxia; gastrointestinal tract disorders involving changes in motility and secretion; negative symptoms of schizophrenia, premenstrual syndrome, fibromyalgia syndrome, stress incontinence, Tourette's syndrome, trichotillomania, kleptomania, male impotence, cancer, small cell lung carcinoma, chronic paroxysmal hemicrania, headache associated with vascular disorders, autism, pervasive developmental disorder NOS, Asperger's disorder, selective mutism, chronic motor or vocal tic disorder, somatization disorder, insomnia, intermittent explosive disorder, pyromania, pathological gambling, impulse-control disorder, premenstrual dysphoric disorder and attention-deficit/hyperactivity disorder (ADHD), in a mammal, comprising administering to a mammal requiring such treatment:
a) one or more 5-HT_{1A} antagonists or a pharmaceutically acceptable salt thereof; and
b) one or more 5-HT_{1D} antagonists according to claim 1 or a pharmaceutically acceptable salt thereof;
wherein the amounts of the active compounds are such that the combination is effective in treating such disorder or condition.

18. A pharmaceutical composition for treating a disorder or condition that can be treated by enhancing serotonergic neurotransmission in a mammal, comprising:
a) one or more 5-HT_{1A} antagonists or a pharmaceutically acceptable salt thereof; and
b) one or more 5-HT_{1D} antagonists according to claim 1 or a pharmaceutically acceptable salt thereof;
and a pharmaceutically acceptable carrier;
wherein the amounts of the active compounds are such that the combination is effective in treating such disorder or condition.
